(19) United States
(12) Patent Application Publication
(10) Pub. No.: US 2013/0095376 A1
(43) Pub. Date: Apr. 18, 2013

(54) ELECTRODE ACTIVE MATERIAL FOR SECONDARY BATTERY AND SECONDARY BATTERY

(71) Applicant: CANON KABUSHIKI KAISHA, Tokyo (JP)

(72) Inventors: Atsuhito Yoshizawa, Kawasaki-shi (JP); Taketoshi Okubo, Asaka-shi (JP)

(73) Assignee: CANON KABUSHIKI KAISHA, Tokyo (JP)

(21) Appl. No.: 13/652,218

(22) Filed: Oct. 15, 2012

(30) Foreign Application Priority Data
Oct. 17, 2011 (JP) 2011-227972

(51) Int. Cl. H01M 4/60; C07D 211/94; H01M 10/056

(52) U.S. Cl. 429/188; 429/213; 546/245

(57) ABSTRACT

An electrode active material for a secondary battery includes a radical compound represented by formula (1):

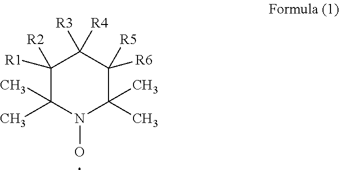

(wherein at least one of R1 to R6 is a protic hydrophilic group); and an alkali metal or an alkaline earth metal.

### BACKGROUND OF THE INVENTION

1. Field of the Invention

The present invention relates to an electrode active material for a secondary battery and to a secondary battery including the same.

2. Description of the Related Art

With a reduction in the size and realization of higher performance of mobile devices such as mobile phones and digital cameras, realization of higher performance of power storage devices used for these mobile devices has been desired. A typical example of such power storage devices is a secondary battery. Among secondary batteries, lithium-ion secondary batteries in which a lithium transition metal oxide is used as a positive electrode and a carbon material is used as a negative electrode have been widely used as secondary batteries having a high energy density. However, in such lithium-ion secondary batteries, the rate of an intercalation/deintercalation reaction of lithium ions in an electrode is low. Thus, lithium-ion secondary batteries cannot be applied to uses that require a high power, for example, sequential shooting using a digital camera with flash light emission.

Electric double-layer capacitors in which activated carbon is used as an electrode have been widely studied as a power storage device having an excellent high-power characteristic.

However, the storage capacity per unit volume of electric double-layer capacitors is low, and thus such electric double-layer capacitors are not suitable for power storage devices for mobile devices, for which a reduction in size has been desired. Therefore, novel secondary batteries that realize both a high capacity and a high power have been studied.

Japanese Patent Laid-Open No. 2002-151084 discloses a secondary battery that utilizes an oxidation-reduction reaction of a stable radical. In this secondary battery, a radical compound (2,2,6,6-tetramethylpiperidine1-oxyl, hereinafter may be abbreviated as “TEMPO”) represented by formula (6) below is used as an active material of a positive electrode or a negative electrode.

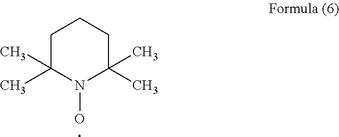

This active material TEMPO consists of elements having low specific gravities, such as carbon and nitrogen. Furthermore, in radical compounds, since reactive unpaired electrons are locally present in radical atoms, the concentration of a reaction site can be increased. Accordingly, realization of high capacity of the electrode can be expected. Furthermore, since only the radical site contributes to a reaction, it is possible to provide a highly stable secondary battery whose cycle characteristics do not depend on the diffusion of the active material. Furthermore, since the structure of the compound does not change in this oxidation-reduction process, the rate of the oxidation-reduction reaction is high and a high power can be expected.

However, such a secondary battery using TEMPO has the following problem.

In order to widen the potential window, in general, organic solvents are used as a solvent of an electrolyte solution in an electrolyte of a secondary battery. However, TEMPO is an organic substance, and thus dissolves in typical organic solvents used as a solvent of an electrolyte.

In order to solve this problem, Japanese Patent Laid-Open No. 2002-304996 discloses an electrode active material and a secondary battery whose resistance to dissolution in electrolyte solutions is enhanced by polymerizing TEMPO.

However, the secondary battery described in Japanese Patent Laid-Open No. 2002-304996 still has the following problem.

In the case where a radical compound is used as an electrode active material, electrical conductivity is supplemented by adding a conductive material to an electrode as in the case of typical lithium-ion secondary batteries. However, in the case where TEMPO is polymerized as in Japanese Patent Laid-Open No. 2002-304996, a contact property between a TEMPO site and the conductive material decreases, and thus it is necessary to add a larger amount of conductive material. As a result, the capacity of the electrode may be decreased on the whole.

### SUMMARY OF THE INVENTION

The present invention provides, as an electrode active material for a secondary battery, a low-molecular-weight radical compound which has an improved resistance to dissolution in electrolyte solutions as compared with existing low-molecular-weight radical compounds and which can realize an electrode for a secondary battery having a larger storage capacity per unit volume than that of the above-described polymer compound.

An electrode active material for a secondary battery according to an aspect of the present invention includes a radical compound represented by formula (1):

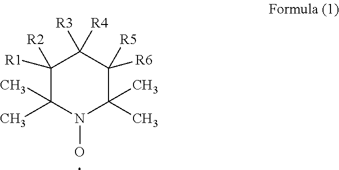

(wherein at least one of R1 to R6 is a protic hydrophilic group); and an alkali metal or an alkaline earth metal.

Further features of the present invention will become apparent from the following description of exemplary embodiments with reference to the attached drawings.

### BRIEF DESCRIPTION OF THE DRAWINGS

FIG. 1 is a schematic view of an oxidation-reduction reaction between a neutral radical and a cation.

FIG. 2 is a schematic view of an oxidation-reduction reaction between a neutral radical and an anion.

FIG. 3 is a schematic view illustrating an example of the structure of a secondary battery.

FIG. 4 includes cyclic voltammograms of secondary batteries of Examples 1 and 2 and Comparative Example 1.

### DESCRIPTION OF THE EMBODIMENTS

The inventors of the present invention have examined, as an electrode active material for a secondary battery, the electrode active material utilizing an oxidation-reduction reaction of a radical, radical compounds that can realize both resistance to dissolution in electrolyte solutions and improvement in a contact property between the radical compound and a conductive material. As a result of the examinations, it was found that an electrode active material for a secondary battery, the electrode active material including a low-molecular-weight radical compound represented by formula (1) below and an alkali metal or an alkaline earth metal has a higher resistance to dissolution in electrolyte solutions than that of the radical compound (TEMPO) represented by formula (6) above. In formula (1) below, at least one of R1 to R6 is a protic hydrophilic group. In the present invention, the term “alkaline earth metal” refers to so-called alkaline earth metals in a broad sense, and refers to group II elements including beryllium and magnesium. In the present invention, the term “hydrophilic group” refers to a concept that also includes an ionized hydrophilic group.

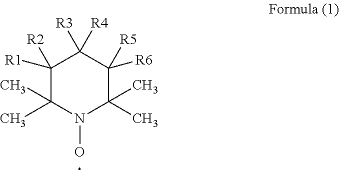

The radical compound represented by formula (1) is a compound in which, among hydrogen atoms (H) at the 3-position, the 4-position, and the 5-position of TEMPO, at least one hydrogen atom is substituted with a protic hydrophilic group. Two hydrogen atoms at the same position may each be substituted with a protic hydrophilic group. The electrode active material including a radical compound represented by formula (1) and an alkali metal or an alkaline earth metal is considered to be a metal salt of a TEMPO derivative, the metal salt being formed by the radical compound represented by formula (1) and the alkali metal or the alkaline earth metal. This electrode active material does not easily dissolve in an electrolyte solution of a secondary battery, though this electrode active material has a low molecular weight. Accordingly, this electrode active material functions as an electrode active material of a secondary battery including an electrolyte solution as an electrolyte. Furthermore, since the electrode active material including a radical compound represented by formula (1) and an alkali metal or an alkaline earth metal has a low molecular weight, this electrode active material has a good contact property with a conductive material, as compared with polymerized radical compounds. When the electrode active material has a good contact property with a conductive material, the amount of conductive material added can be reduced. Accordingly, a decrease in the capacity caused by adding a conductive material to an electrode can be suppressed, and the capacity of the whole electrode can be made larger than that in the case where a polymerized radical compound is used.

The present invention will be described in more detail using embodiments of an electrode active material for a secondary battery of the present invention and embodiments of a secondary battery including the electrode active material.

### Electrode Active Material for Secondary Battery

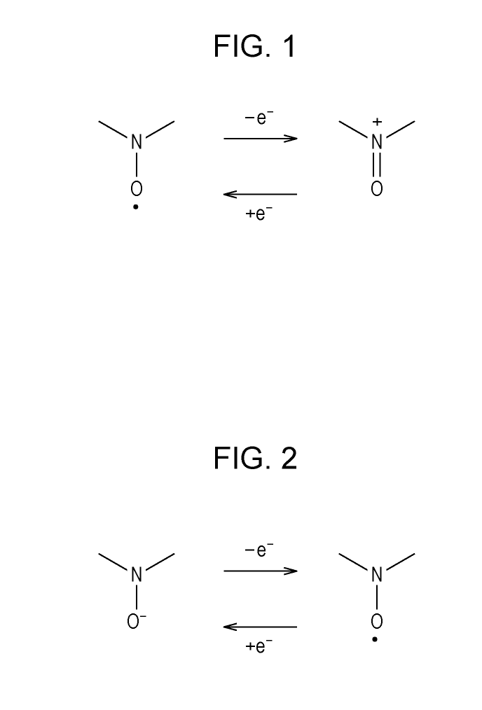

An electrode active material according to this embodiment includes a radical compound represented by formula (1) and an alkali metal or an alkaline earth metal. As described above, at least one of R1 to R6 in formula (1) is a protic hydrophilic group. The electrode active material according to this embodiment can store and release an electron through an oxidation-reduction reaction between a neutral radical and a cation as illustrated in FIG. 1. The position and the type of protic hydrophilic group and the type of alkali metal or alkaline earth metal are not particularly limited. Embodiments of the protic hydrophilic group and the alkali metal or the alkaline earth metal will be described below.

### (Protic Hydrophilic Group)

The protic hydrophilic group in the radical compound according to this embodiment is converted to an anion by releasing a proton in water at a specific pH. Examples of the protic hydrophilic group include —COOH, —S(═O)₂OH, and —P(═O)(OH)₂. The type of protic hydrophilic group is not particularly limited as long as hydrophilicity is exhibited. However, the lower the molecular weight of the electrode active material, the lower the molecular weight per unit radical, and thus the larger the storage capacity (mAh/g) of the active material. Accordingly, —COOH, which has a low molecular weight, is preferably used as the protic hydrophilic group. Among R1 to R6, the position of the protic hydrophilic group is not particularly limited. Furthermore, the number of protic hydrophilic groups in R1 to R6 is also not particularly limited. However, from the standpoint of the molecular weight described above, only any one of R1 to R6 is preferably a protic hydrophilic group and R1 to R6 other than the protic hydrophilic group are each preferably a hydrogen atom. In the case where a carboxyl group, which has a strong electron-withdrawing property, is located at the 4-position (position of R3 or R4), the nitroxyl group is stabilized in an anion state. As a result, not only the oxidation-reduction reaction between a neutral radical and a cation illustrated in FIG. 1 but also an oxidation-reduction reaction between a neutral radical and an anion illustrated in FIG. 2 stably proceeds. Accordingly, a hydrogen atom at the 4-position is more preferably substituted with a carboxyl group. As in the case of the “hydrophilic group” described above, the term “carboxyl group” in the present invention refers to a concept including an ionized carboxyl group (i.e., a carboxyl group from which a proton has been released).

### (Alkali Metal or Alkaline Earth Metal)

As described above, it is believed that an alkali metal or an alkaline earth metal in the electrode active material according to this embodiment forms a salt with the radical compound represented by formula (1). The alkali metal or the alkaline earth metal is not particularly limited. However, as described above, the lower the molecular weight of the electrode active material, the larger the storage capacity of the electrode active material. Therefore, the molecular weight of the alkali metal or the alkaline earth metal is also preferably low. Accordingly, the alkali metal or the alkaline earth metal in the electrode active material is preferably Li, Na, Mg, or Ca.

In the case where an ion of an alkali metal or an ion of an alkaline earth metal is present in an electrolyte of a secondary battery, the alkali metal or the alkaline earth metal contained in the electrode active material is preferably the alkali metal or alkaline earth metal that forms the ion present in the electrolyte.

When an ion of the alkali metal or alkaline earth metal contained in the electrode active material is present in the electrolyte, the electrode active material does not more easily dissolve in the electrolyte. For example, in a lithium-ion secondary battery, an electrolyte solution obtained by dissolving a lithium salt in an organic solvent is widely used. In the case where such an electrolyte solution is used, the electrode active material preferably includes the radical compound represented by formula (1) and Li.

### (R1 to R6)

As long as at least one of R1 to R6 is a protic hydrophilic group, R1 to R6 other than the protic hydrophilic group are not particularly limited. However, when R1 to R6 are hydrophobic substituents, hydrophilicity of the radical compound represented by formula (1) decreases and the solubility of the electrode active material in organic solvents increases. Thus, the solubility of the electrode active material in electrolyte solutions containing organic solvents also increases. That is, resistance to dissolution of the electrode active material in electrolyte solutions decreases. Accordingly, preferably, the compound represented by formula (1) does not have a hydrophobic substituent.

In the present invention, since R1 to R6 are hydrophilic substituents, hydrophilicity of the radical compound represented by formula (1) increases, and resistance to dissolution of the electrode active material in electrolyte solutions decreases. Furthermore, it is believed that when R1 to R6 are protic hydrophilic groups, a salt can be formed with an alkali metal or an alkaline earth metal.

As described in the above paragraph regarding protic hydrophilic groups, from the standpoint of the molecular weight, R1 to R6 are each preferably a substituent having a low molecular weight or a hydrogen atom. Three or more of R1 to R6 are each preferably a hydrogen atom. Furthermore, more preferably, only one of R1 to R6 is a protic hydrophilic group and the remaining five groups are each a hydrogen atom.

Specifically, for example, a radical compound having, as R1 to R6, one protic hydrophilic group, two hydrophilic groups other than protic hydrophilic groups, and three hydrogen atoms is more preferable than a radical compound having, as R1 to R6, one protic hydrophilic group, two hydrophobic groups, and three hydrogen atoms. A radical compound having, as R1 to R6, three protic hydrophilic groups and three hydrogen atoms is more preferable. A radical compound having, as R1 to R6, one protic hydrophilic group and five hydrogen atoms is more preferable than such a radical compound having three protic hydrophilic groups and three hydrogen atoms.

### Secondary Battery

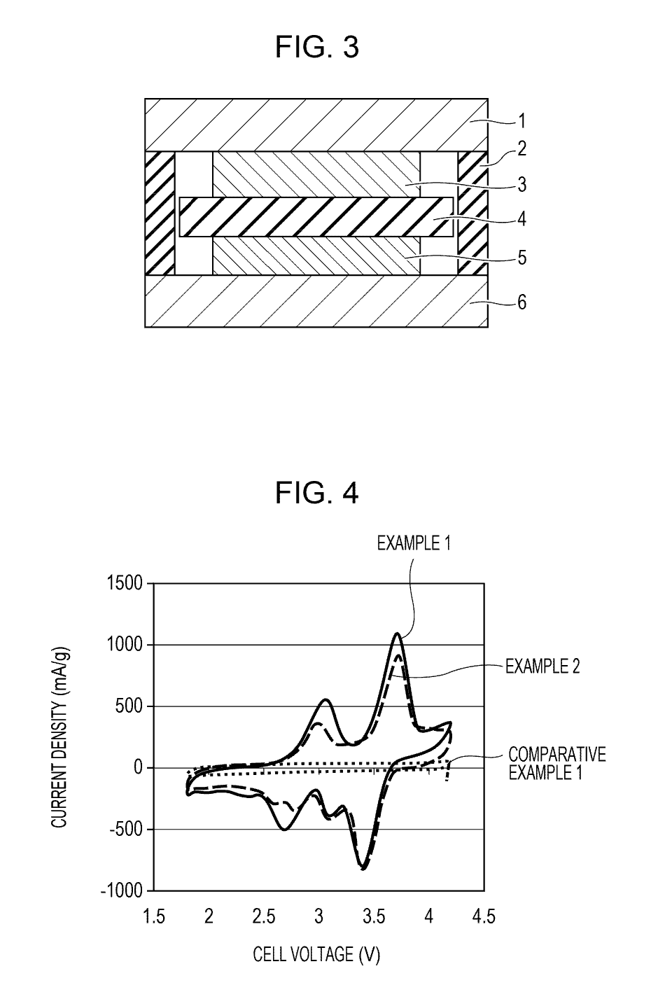

A secondary battery of this embodiment includes at least a positive electrode, a negative electrode, and an electrolyte. FIG. 3 illustrates an example of the structure of the secondary battery. The secondary battery illustrated in the figure has a structure in which a positive electrode current collector 1, a positive electrode 3, a separator 4, a negative electrode 5, and a negative electrode current collector 6 are sequentially stacked. The separator 4 contains an electrolyte. The positive electrode current collector 1 and the negative electrode current collector 6 are insulated from each other with an insulating packing 2 composed of a plastic resin therebetween. A polymer gel electrolyte may also be used instead of the separator containing an electrolyte. The positive electrode 3 can also be referred to as “positive electrode material layer” or “positive electrode active material layer”. The negative electrode 5 can also be referred to as “negative electrode material layer” or “negative electrode active material layer”.

The form of the secondary battery may be a known form. For example, the secondary battery may have a form in which a laminate or a wound body of electrodes is sealed in a metal case, a resin case, a laminated film, or the like. Examples of the appearance of the secondary battery include a cylindrical shape, a rectangular parallelepiped shape, a coin shape, and a sheet shape.

Each component of the secondary battery of this embodiment will now be described.

### (Positive Electrode)

In the secondary battery of this embodiment, the active material for a secondary battery according to this embodiment is used as at least an electrode active material of the positive electrode or the negative electrode. In the case where the active material according to this embodiment is used in only one of the positive electrode and the negative electrode, the active material is preferably used in the positive electrode. In addition to the electrode active material according to this embodiment, known other components may be incorporated in the positive electrode. Examples of the known components include a conductive material and a binder (binding agent). Examples of the conductive material include carbon materials such as activated carbon, graphite, carbon black, and acetylene black; and conductive polymers such as polyacetylene, polyphenylene, polyaniline, and polypyrrole. Examples of the binder include resin binders such as polyvinylidene fluoride, polytetrafluoroethylene, polyvinylidene fluoride-hexafluoropropylene copolymers, styrene-butadiene copolymer rubber, polypropylene, polyethylene, and polyimide; and ion-conductive polymers. These components can be appropriately incorporated.

In the case where the active material for a secondary battery according to this embodiment is used only in the negative electrode, known active materials can be used as the positive electrode. Examples of the active material that can be used include particles of a metal oxide such as lithium cobalt oxide or lithium manganese oxide; disulfide compounds; and conductive polymers such as polyacetylene, polyphenylene, polyaniline, and polypyrrole. Known active materials and the active material of this embodiment may be used in combination.

### (Negative Electrode)

In the case where the electrode active material according to this embodiment is used as the active material of the negative electrode, in addition to the electrode active material according to this embodiment, known other components may be incorporated in the negative electrode, as in the case of the positive electrode.

In the case where the electrode active material for a secondary battery according to this embodiment is used only in the positive electrode, known electrode active materials can be used as the negative electrode. Examples of the electrode active material that can be used include carbon materials such as activated carbon, graphite, carbon black, and acetylene black, lithium metal, lithium alloys, lithium ion-occluding carbon, tin metal, tin alloys, silicon metal, silicon alloys, other elemental metals, alloys thereof, and conductive polymers such as polyacetylene, polyphenylene, polyaniline, and polypyrrole. Furthermore, other components such as resin binders, e.g., polyvinylidene fluoride, polytetrafluoroethylene, a polyvinylidene fluoride-hexafluoropropylene copolymer, styrene-butadiene copolymer rubber, polypropylene, polyethylene, and polyimide; and ion-conductive polymers can also be appropriately incorporated.

### (Current Collector)

The materials of the positive electrode current collector 1 and the negative electrode current collector 6 are not particularly limited as long as the materials have high electrical conductivity and good corrosion resistance. Examples thereof include metals such as nickel, aluminum, copper, gold, silver, and titanium; alloys such as aluminum alloys and stainless steel, and carbon materials. Examples of the shape of the current collectors include a foil, a flat plate, and a mesh.

### (Separator)

The secondary battery of this embodiment includes the separator 4 for the purpose of preventing electrical contact between the positive electrode 3 and the negative electrode 5. For example, a non-woven fabric or a separator composed of a porous film can be used as the separator. An electrolyte described below is contained in the separator of this embodiment.

### (Electrolyte)

The secondary battery of this embodiment includes an electrolyte. The electrolyte conducts the transport of a charge carrier between the negative electrode and the positive electrode, and generally has an electrolyte ion conductivity of 10⁻⁵ to 10⁻¹ S/cm at room temperature. For example, an electrolyte solution prepared by dissolving an electrolyte salt in a solvent can be used as the electrolyte in this embodiment. The solvent of the electrolyte solution is not particularly limited as long as the solvent has a wide potential window and can ionize the electrolyte salt. Examples thereof include organic solvents such as ethylene carbonate, propylene carbonate, dimethyl carbonate, diethyl carbonate, methylethyl carbonate, γ-butyrolactone, tetrahydrofuran, dioxolane, sulfolane, dimethylformamide, dimethylacetamide, and N-methyl-2-pyrrolidone. These solvents may be used alone or in combination of two or more solvents.

Examples of the electrolyte salt include LiPF₆, LiClO₄, LiBF₄, LiCF₃SO₃, LiN(CF₃SO₂)₂, LiN(C₂F₅SO₂)₂, LiC(CF₃SO₂)₃. LiC(C₂F₅SO₂)₃, LiBr, LiCl, and LiF.

In the structure of the secondary battery illustrated in FIG. 3, the separator 4 containing an electrolyte is used.

Instead of the electrolyte solution described above, for example, a solid electrolyte, which is a gelled electrolyte solution, may also be used. In this case, the resulting secondary battery is a so-called polymer secondary battery. Even in the case where a gelled electrolyte is used, when an organic solvent is contained in the electrolyte and an electrode active material having a high solubility in the organic solvent is used, the electrode active material may dissolve from an electrode to the electrolyte. However, the use of the electrode active material according to this embodiment can suppress the dissolution of the electrode active material in the electrolyte.

### EXAMPLES

More specific Examples of the above embodiments will now be described.

### Preparation of Electrode Active Material A

In 50 cc of ion-exchange water, 0.12 g (0.005 mol) of lithium hydroxide (manufactured by Tokyo Chemical Industry Co., Ltd.) was dissolved to prepare a 0.1 M aqueous lithium hydroxide solution. Subsequently, 1 g (0.005 mol) of 4-carboxy-2,2,6,6-tetramethylpiperidine1-oxyl free radical (manufactured by Tokyo Chemical Industry Co., Ltd.) represented by formula (2) was added thereto and dissolved in the solution by stirring. The resulting solution was dried under vacuum at 60° C. for eight hours to obtain an electrode active material A. It is believed that the electrode active material A is a salt of a radical compound represented by formula (2) and lithium, as shown in formula (3).

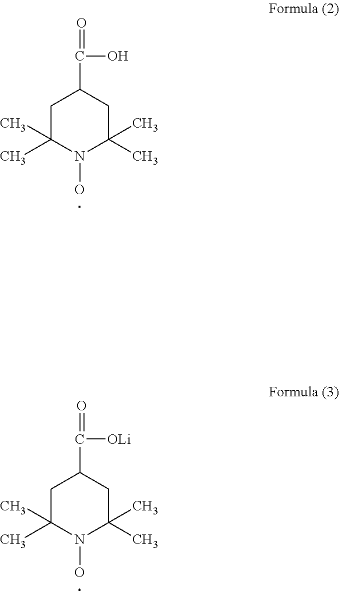

### Preparation of Electrode Active Material B

To 5 cc of a 1 M aqueous sodium hydroxide solution (manufactured by Tokyo Chemical Industry Co., Ltd.), 45 cc of ion-exchange water was added to prepare a 0.1 M aqueous sodium hydroxide solution. Subsequently, 1 g (0.005 mol) of 4-carboxy-2,2,6,6-tetramethylpiperidine1-oxyl free radical (manufactured by Tokyo Chemical Industry Co., Ltd.) represented by formula (2) was added thereto and dissolved in the solution by stirring. The resulting solution was dried under vacuum at 60° C. for eight hours to obtain an electrode active material B. It is believed that the electrode active material B is a salt of the radical compound represented by formula (2) and sodium, as shown in formula (4).

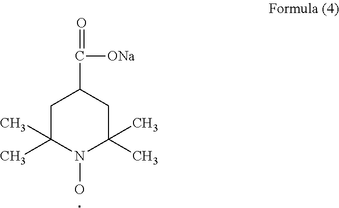

### Preparation of Electrode Active Material C

In 50 cc of ion-exchange water, 0.185 g (0.0025 mol) of calcium hydroxide (manufactured by Kishida Chemical Co., Ltd.) was dissolved to prepare a 0.05 M aqueous calcium hydroxide solution. Subsequently, 1 g (0.005 mol) of 4-carboxy-2,2,6,6-tetramethylpiperidine1-oxyl free radical (manufactured by Tokyo Chemical Industry Co., Ltd.) represented by formula (2) was added thereto and dissolved in the solution by stirring. The resulting solution was dried under vacuum at 60° C. for eight hours to obtain an electrode active material C. It is believed that the electrode active material C is a salt of the radical compound represented by formula (2) and calcium, as shown in formula (5).

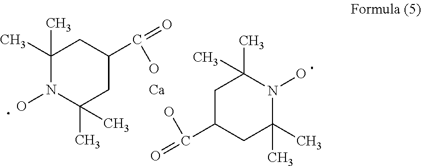

### Preparation of Positive Electrode

First, 2.4 g of an electrode active material (any of the electrode active materials A to C), 1.2 g of acetylene black (manufactured by Denki Kagaku Kogyo Kabushiki Kaisha) serving as a conductive material, 0.4 g a polyvinylidene fluoride-hexafluoropropylene copolymer (manufactured by Aldrich) serving as a binder, and 4 cc of N-methylpyrrolidone (manufactured by Kishida Chemical Co., Ltd.) were mixed, and the resulting mixture was stirred using a planetary ball mill at 150 rpm for 30 minutes to prepare a slurry. The slurry was applied onto an aluminum foil with a blade coater, and was dried under vacuum at 80° C. for eight hours to remove N-methylpyrrolidone. Thus, an electrode sheet (integrated component of the positive electrode 3 and the positive electrode current collector 1) was prepared. Regarding this electrode sheet, one electrode sheet was prepared for each of the electrode active materials A, B, and C. Thus, a total of three electrode sheets were prepared.

### Preparation of Electrolyte

An electrolyte solution was prepared by dissolving 152 g (1 mol) of LiPF₆ (manufactured by Kishida Chemical Co., Ltd.) functioning as an electrolyte salt in a mixed solvent containing 300 cc of ethylene carbonate (manufactured by Kishida Chemical Co., Ltd.) and 700 cc of diethyl carbonate (manufactured by Kishida Chemical Co., Ltd.). A porous separator was impregnated with the prepared electrolyte solution for eight hours to prepare a separator containing an electrolyte.

### Preparation of Secondary Battery

The positive electrode prepared as above was punched so as to have a diameter of 8 mm, the separator containing the electrolyte was punched so as to have a diameter of 12 mm, and a lithium metal foil functioning as a negative electrode was so as to have a diameter of 10 mm. These electrodes and the separator were stacked so that the separator containing the electrolyte was sandwiched between the positive electrode and the negative electrode. Thus, laminates were prepared. The laminates were set in an HS cell (trade name), which is an experimental cell manufactured by Hohsen Corporation. Thus, secondary batteries were prepared.

### Example 1

The electrode active material A was used as an electrode active material of the positive electrode. The weight of the prepared electrode sheet was 5 mg/cm² except for the current collector portion.

### Example 2

The electrode active material B was used as an electrode active material of the positive electrode. The weight of the prepared electrode sheet was 5 mg/cm² except for the current collector portion.

### Example 3

The electrode active material C was used as an electrode active material of the positive electrode. The weight of the prepared electrode sheet was 5 mg/cm² except for the current collector portion.

### Comparative Example 1

A secondary battery was prepared as in Examples 1 to 3 except that the electrode active material represented by formula (6) was used as an electrode active material of the positive electrode. The weight of the prepared electrode sheet was 5 mg/cm² except for the current collector portion.

### Comparative Example 2

A secondary battery was prepared as in Examples 1 to 3 except that the electrode active material represented by formula (2) was used as an electrode active material of the positive electrode. The weight of the prepared electrode sheet was 5 mg/cm² except for the current collector portion.

Battery characteristics of the secondary batteries of Examples 1 to 3 and Comparative Examples 1 and 2 prepared as described above were evaluated. FIG. 4 includes cyclic voltammograms of the secondary batteries of Examples 1 and 2 and Comparative Example 1. The scanning speed was set to 1 mV/s, and the scanning range was set to 4.2 to 1.8 V. Regarding the secondary battery of Comparative Example 1, the electrode active material represented by formula (6) was eluted in the electrolyte solution, and no charge/discharge peaks were observed. In contrast, regarding the secondary batteries of Examples 1 and 2, redox couples were observed at about 3.6 V and 3.4 V, and 2.8 V and 2.6 V, showing that the electrode active materials were not eluted in the electrolyte solution. The electrode active materials A and B of Examples 1 and 2 differ from the electrode active material of Comparative Example 1 represented by formula (6) in that a carboxyl group, which is a protic hydrophilic group, is provided to the radical compound and the electrode active materials have Li or Na (a Li ion or a Na ion). Accordingly, it is believed that this difference in the structure improves the resistance to dissolution in electrolyte solutions, and as a result, the elution of the electrode active materials in the electrolyte solution is suppressed.

The open circuit voltages (OCV) of the secondary batteries of Examples 1 and 2 before the cyclic voltammetry were each 3.2 V. Accordingly, it is believed that the redox couple at 3.6 V and 3.4 V, the redox couple being observed at voltages higher than the OCV, is attributable to an oxidation-reduction reaction between a neutral radical and a cation illustrated in FIG. 1. It is believed that the redox couple at 2.8 V and 2.6 V, the redox couple being observed at voltages lower than the OCV, is attributable to an oxidation-reduction reaction between a neutral radical and an anion illustrated in FIG. 2. In general, a nitroxyl group is unstable in the state of an anion, and the oxidation-reduction reaction between the neutral radical and the anion illustrated in FIG. 2 does not easily proceed. It is known that, however, when an electron-withdrawing group is present at the 2-position or the 4-position of a nitroxyl group, the nitroxyl group has a stable structure in the state of an anion. It is believed that each of the electrode active material A in the secondary battery of Example 1 and the electrode active material B in the secondary battery of Example 2 has a carboxyl group, which is an electron-withdrawing group, at the 4-position of a nitroxyl group, and thus the nitroxyl group is stabilized in the state of an anion.

Next, in order to quantitatively evaluate the resistance to dissolution of the electrode active materials of the present invention in electrolyte solutions, a constant-current charge/discharge test was conducted using the secondary batteries of Examples 1 and 3 and Comparative Example 2. The evaluation method is as follows: The positive electrode capacity immediately after the preparation of each secondary battery and the positive electrode capacity after the secondary battery was left to stand for 24 hours from the preparation were measured. The capacities of the electrode active material were calculated and compared with each other to evaluate the elution of the electrode active material in the electrolyte solution. The voltage range in the constant-current charge/discharge test was set to a range of 2.8 to 3.8 V in order to cause an oxidation-reduction reaction between a neutral radical and a cation illustrated in FIG. 1 to proceed. The current density was calculated from a theoretical capacity of each electrode active material and the weight of the electrode and determined so as to be 5 C (so that charging and discharging were completed in ⅕ hours=12 minutes). Specifically, the charge/discharge test of the secondary battery of Example 1 was conducted with a current of 0.9075 mA (=1.815 mA/cm²). The charge/discharge test of the secondary battery of Example 3 was conducted with a current of 0.855 mA (=1.71 mA/cm²). The charge/discharge test of the secondary battery of Comparative Example 2 was conducted with a current of 0.93 mA (=1.86 mA/cm²). The results of the charge/discharge test are shown in Table 1. Regarding the secondary battery of Example 1, the theoretical capacity of the electrode active material A was 121 mAh/g, and the capacity of the electrode active material A in the positive electrode immediately after the preparation of the battery was 118 mAh/g, which was substantially the same as the theoretical capacity. Furthermore, regarding the test result obtained after the battery was left to stand for 24 hours from the preparation of the battery, the capacity of the electrode active material A was 116 mAh/g, and thus a decrease in the capacity was hardly observed. Even when the positive electrode including the electrode active material A contacted an electrolyte solution for 24 hours, the capacity of the positive electrode did not substantially decrease from the initial capacity and the theoretical capacity. These results show that the elution of the electrode active material A in the electrolyte solution hardly occurred. Similarly, regarding the secondary battery of Example 3, the theoretical capacity of the electrode active material C was 114 mAh/g, and the capacity measured after the battery was left to stand for 24 hours from the preparation of the battery was 110 mAh/g. Similarly to the electrode active material A, these results show that the elution of the electrode active material C did not occur even when the positive electrode including the electrode active material C contacted an electrolyte solution for 24 hours. In contrast, regarding the secondary battery of Comparative Example 2, the theoretical capacity of the electrode active material represented by formula (2) was 124 mAh/g, the capacity immediately after the preparation of the battery was 86 mAh/g, and the capacity after 24 hours from the preparation of the battery was 41 mAh/g. Thus, a decrease in the capacity was observed. These results show that the electrode active material represented by formula (2) started to elute in an electrolyte solution immediately after the preparation of the battery, and the amount of elution was increased with time. In Comparative Example 1, in which the electrode active material represented by formula (6) was used, a charge-discharge reaction did not occur. On the other hand, in Comparative Example 2, in which the electrode active material represented by formula (2) was used, a charge-discharge reaction occurred, though the capacity of the electrode active material was significantly decreased from the theoretical capacity. The electrode active material represented by formula (2) is a material obtained by providing a carboxyl group, which is a protic hydrophilic group, to the electrode active material represented by formula (6). Accordingly, it is believed that the resistance to dissolution in electrolyte solutions was improved by the protic hydrophilic group, and the charge-discharge reaction occurred. However, it is believed that the elution of the electrode active material in the electrolyte solution was caused by the contact with an electrolyte solution for a long time. Accordingly, in order to stably use a radical compound as an electrode active material, it is effective to provide a protic hydrophilic group to the radical compound represented by formula (6) and to allow the radical compound to react with an alkali metal or an alkaline earth metal.

TABLE 1

| | | | Comparative |
|---|---|---|---|
| | Example 1 | Example 3 | Example 2 |
| Theoretical | 121 mAh/g | 114 mAh/g | 124 mAh/g |
| capacity | | | |
| Capacity | 118 mAh/g | | 86 mAh/g |
| immediately | | | |
| after | | | |
| preparation | | | |
| of battery | | | |
| Capacity after | 116 mAh/g | 110 mAh/g | 41 mAh/g |
| 24 hours from | | | |
| preparation of | | | |
| battery | | | |

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2011-227972 filed Oct. 17, 2011, which is hereby incorporated by reference herein in its entirety.

What is claimed is:

1. An electrode active material for a secondary battery, comprising:
a radical compound represented by formula (1): 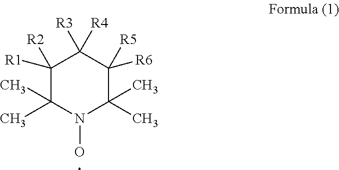
wherein at least one of R1 to R6 is a protic hydrophilic group; and
an alkali metal or an alkaline earth metal.

2. The electrode active material according to claim 1, wherein R1 to R6 are each a hydrogen atom or a protic hydrophilic group.

3. The electrode active material according to claim 1, wherein the protic hydrophilic group is a carboxyl group.

4. The electrode active material according to claim 1, wherein the alkali metal or alkaline earth metal is lithium.

5. A secondary battery comprising:
a positive electrode;
a negative electrode; and
an electrolyte present between the positive electrode and the negative electrode,
wherein at least one of the positive electrode and the negative electrode includes the electrode active material for a secondary battery according to claim 1.

6. The secondary battery according to claim 5, wherein the electrolyte contains an ion of the alkali metal or alkaline earth metal in the electrode active material.
